(19) United States
(12) Patent Application Publication
(10) Pub. No.: US 2022/0346633 A1
(43) Pub. Date: Nov. 3, 2022

(54) AN OPTICAL FIBER CATHETER PROBE AND A MANUFACTURING METHOD THEREOF

(71) Applicant: UNIVERSITY COLLEGE CORK - NATIONAL UNIVERSITY OF IRELAND, CORK, Cork (IE)

(72) Inventor: Peter O'Brien, Cork City (IE)

(21) Appl. No.: 17/779,382

(22) PCT Filed: Nov. 25, 2020
(86) PCT No.: PCT/EP2020/083421

§ 371 Date: May 24, 2022

(30) Foreign Application Priority Data
Nov. 25, 2019 (GB) 1917150.3

(51) Int. Cl. A61B 1/00; A61B 5/00

(52) U.S. Cl. CPC A61B 1/00096; A61B 1/00165; A61B 5/6852; A61B 5/0066

(57) ABSTRACT

The catheter probe comprises a catheter tip and a catheter body. The catheter tip comprises a plurality of channels for housing a plurality of optical sensors. Each one of said plurality of optical sensors comprises a ferrule comprising a via, a microlens aligned with said via and attached to said ferrule and an optical fiber for optically coupling said microlens to a monitoring means.

### FIELD

The present disclosure relates to an optical fiber catheter probe.

### BACKGROUND

Medical devices such as catheters are used for observing internal organs or tissues of a subject. A catheter is usually inserted in the subject's vascular system, where such a catheter is required to be sufficiently long (in the range meters) to traverse through the subject's vascular system. Also, the catheter needs to be flexible to ensure it can bend along the complex atrial network of the subject. Typically examples of optical sensing include monitoring of tissue ablation using heated (RF) assemblies at the tip of the catheter, see FIG. 1. Heating of the tissue or cardiac ablation is a procedure that can correct heart rhythm problems (arrhythmias).

The optical sensor can be used to monitor the tissue ablation process, giving surgeons real-time information about the procedure. Optical probing techniques such as Optical Coherence Tomography (OCT) can be used in these applications. In this case, light is sent from a source, along the catheter and out of the tip of the catheter (near the ablation device which is also integrated on the catheter tip). The reflected optical signal from the tissue is then collected by the sensor component and returned to the source instrument for interrogation and read-out to the surgeon.

However, the present catheter probes do not allow for multiple optical sensors and therefore provide a very limited field of view to a surgeon or a medical practitioner. Also, there is a significant challenge in housing a wide angle optical mechanism due to the limited size and cross-sectional area of the catheter.

U.S. Pat. No. 5,29,275 (MIT) describes a catheter type device with fiber optics and lens, and an optical shield to protect the optics from body fluids.

However a problem with this approach is that it will render the glass lens ineffective as the refractive index of the glass lens matches or is close to that of water/blood. This means the lens loses it's focusing power and hence the need for the optical shield to protect it. Similar drawbacks exist with WO2018/138490 (Smith et al).

Therefore, there is an unfulfilled and unresolved need for a compact catheter probe with an improved/wide field of view.

### SUMMARY

The present invention relates to an optical fiber catheter probe, as set out in the appended claims. More specifically, the present invention relates to an optical fiber catheter probe having a plurality of optical sensors.

In one embodiment there is provided a catheter probe, the probe comprises:
a catheter tip, said catheter tip comprising a plurality of channels or tunnels for housing a plurality of optical sensors, each plurality of optical sensors comprising:
a ferrule comprising a central micro via;
an optical fiber passed through said micro via
a microlens attached with said ferrule and aligned to said optical fiber; and
a catheter body for housing said optical fibers to a monitoring means.

The invention provides a means of manufacturing a highly compact array of optical sensors within a small cross-sectional area and capable of imaging over a large field of view. For example over twenty individual optical fiber sensors can be integrated within a standard catheter. The use of narrow cladding and high numerical aperture optical fiber enables a large number of sensing fibers to be integrated into the catheter body, while the high NA fiber enables the small bending radii required for navigation through the vascular system. The manufacturing process enables automated flipchip assembly of the optical components (micro lenses) with the micron precision required for the production of single mode optical systems.

In one embodiment the invention uses one or more high index silicon microlenses adapted to be placed directly in the body and still retain its focusing power. This results in a much more compact design which is also less prone to spurious reflections from the protective optical shield which can induce signal errors in the readings. Also, due to its high refractive index, silicon lenses are small which can result in more sensing elements being placed at the catheter tip.

In one embodiment said optical fibre comprises a dimensioned tapered channel to define a fiber mode adapter. The fiber comprises a narrow core fiber to a larger core fiber wherein the tapered channel expands a mode from the narrow core fiber to the larger core fiber.

The narrow core optical fiber running along the catheter can be difficult to align to the microlens (for example a typical fiber core diameter is only 3 microns whereas normal large core fiber is usually 10 microns). The invention provides a ‘mode adaptor element’ to transition between the narrow core fiber and microlens, which increases light coupling efficiency and easy of manufacturing as the mechanical alignment tolerances are reduced.

In one embodiment, said optical fibers have a diameter of about 50 microns and the core diameters of said optical fibers are about 2-5 microns.

In one embodiment, the vias have a diameter of 52-55 microns.

In one embodiment, said optical fibers are configured to operate at wavelengths of around 1310 nanometre.

In a preferred embodiment, the optical fibers are single mode and high numerical aperture fibers.

In another embodiment there is provided a method for manufacturing said optical fiber catheter probe comprises the following steps of:
etching a glass substrate to form a plurality of vias;
inserting an optical fiber in each of said plurality of vias;
polishing a top surface of the glass substrate;
aligning a micro-lens array, where each microlens of the microlens array is aligned with each optical fiber core on said polished top surface of the glass substrate;
bonding said aligned microlens array on said polished top surface of the glass substrate;
dicing said microlens, optical fiber and glass substrate assembly to obtain individual optical sensors; and
inserting each optical sensor into a catheter tip.

In one embodiment, said optical fibers have a diameter of about 50 microns and the core diameters of said optical fibers are about 2-5 microns.

In one embodiment, the vias have a diameter of 52-55 microns.

In one embodiment, said optical fibers are configured to operate at wavelengths of around 1310 nanometre.

In one embodiment, the microlens array is bonded to the top surface of the glass substrate using an ultraviolet cure epoxy.

In a preferred embodiment, the microlens array is made up of silicon.

In another preferred embodiment, the optical fibers are single mode and high numerical aperture fibers.

In another embodiment of the invention there is provided a method for manufacturing a catheter probe comprises the following steps:
etching a glass substrate to form a plurality of vias;
inserting an optical fiber in each of said plurality of vias;
forming a microlens on each optical fiber projecting through said plurality of vias;
dicing glass substrate assembly comprising said glass substrate and said optical fiber and a microlens formed thereon, to obtain individual optical sensors; and
inserting each optical sensor into a catheter tip.

It will be appreciated that the method to process components allows for production in volume rather than part-by-part. Essentially, arrays of devices at wafer-level can be made and singulated after all the challenging mechanical alignment processes have ben done. This avoids a slow part-by-part manufacturing process which is essential for large-scale manufacturing.

In one embodiment, said microlens on each optical fiber is formed using three dimensional polymer stereo-lithography or laser micromachining.

In one embodiment, said microlens on each optical fiber is formed using three dimensional stereo-lithography or laser micromachining on a glass substrate and selective etching of glass structures.

In another embodiment there is provided a method for manufacturing a catheter probe, comprising:
etching a glass substrate to form a plurality of vias;
inserting an optical fiber in each of said plurality of vias;
polishing a top surface of the glass substrate;
aligning a micro-lens array, where each microlens of the microlens array is aligned with each via on said polished top surface of the glass substrate;
bonding said aligned microlens array on said polished top surface of the glass substrate; and
inserting each optical sensor into a catheter tip.

In another embodiment there is provided a method to connect the narrow cladding optical fibers on the catheter to another location or device, such as a measuring instrument. The method be implemented using the following steps:
1) The narrow cladding fibers on the catheter (50 micron cladding diameter, for example) are fusion (heat) spliced to the standard 125 micron (or other dimension) cladding single more fiber which has a core of 10 microns (eg. SMF28 fiber). This enables a smooth and relatively low loss light path between the narrow core and large core optical fibers;
2) The spliced large core optical fibers are then inserted into a precision fiber array connector such as an MPT or MPO connector; and
3) This connector can be easily connected (pluggable style) to optical fibers on the instrument side.

In another embodiment there is provided a catheter probe, comprising:
a catheter tip, said catheter tip comprising at least one channel which housing at least one optical sensor, said optical sensor comprising:
a ferrule comprising a central micro via;
an optical fiber passed through said micro via
a microlens attached to said ferrule and aligned to said optical fiber; and
a catheter body configured to house said optical fiber and connected to a monitoring means.

In one embodiment said optical fibre comprises a dimensioned tapered channel to define a fiber mode adapter. The fiber comprises a narrow core fiber to a larger core fiber wherein the tapered channel expands a mode from the narrow core fiber to the larger core fiber. This fiber mode adapter configuration can be particularly effective in a single sensor embodiment

The invention will now be described in more detail with reference to a preferred embodiment thereof and also with reference to the accompanying drawings.

### BRIEF DESCRIPTION OF THE DRAWINGS

The invention will be more clearly understood from the following description of an embodiment thereof, given by way of example only, with reference to the accompanying drawings, in which:—

FIG. 1 exemplarily illustrates a catheter probe placed inside an organ of a subject;

FIGS. 2A and 2B and exemplarily illustrate a method of manufacture of a catheter probe in accordance with some of the embodiments of the present invention;

FIG. 3 exemplarily illustrates a sectional view of the catheter probe in accordance with some of the embodiments of the present invention;

FIG. 4 exemplarily illustrates another sectional view of the catheter probe in accordance with some of the embodiments of the present invention;

FIG. 5A exemplarily illustrates a sectional view of the catheter tip (cut along the broken line as shown in FIG. 4) of said catheter probe in accordance with some of the embodiments of the present invention;

FIG. 5B exemplarily illustrates a sectional view of the catheter body of said catheter probe in accordance with some of the embodiments of the present invention;

FIG. 6 exemplarily illustrates a sectional view of the catheter body of said catheter probe in accordance with another embodiment of the invention;

FIG. 7 illustrates a mode adaptor design in the optic fiber to transition between a narrow core and standard core optical fiber; and

FIG. 8 illustrates how self-aligning features on a microlens can be achieved to facilitate passive alignment of the microlens to a standard optical fiber, according to one embodiment of the invention.

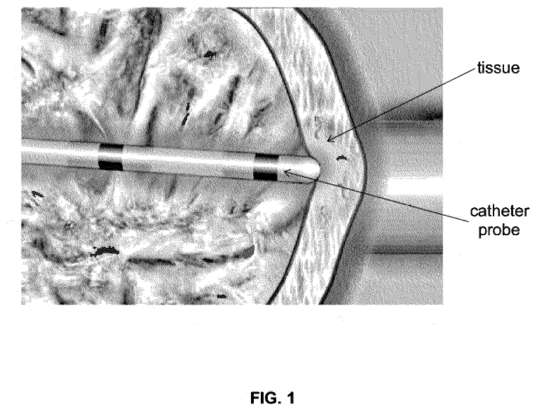

### DETAILED DESCRIPTION OF THE DRAWINGS

The present invention relates to an optical fiber catheter probe. More specifically, the present invention relates to an optical fiber catheter probe having a plurality of optical sensors.

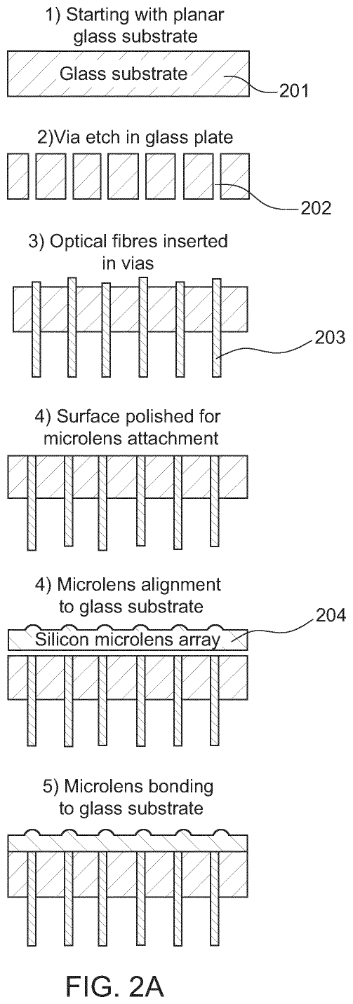

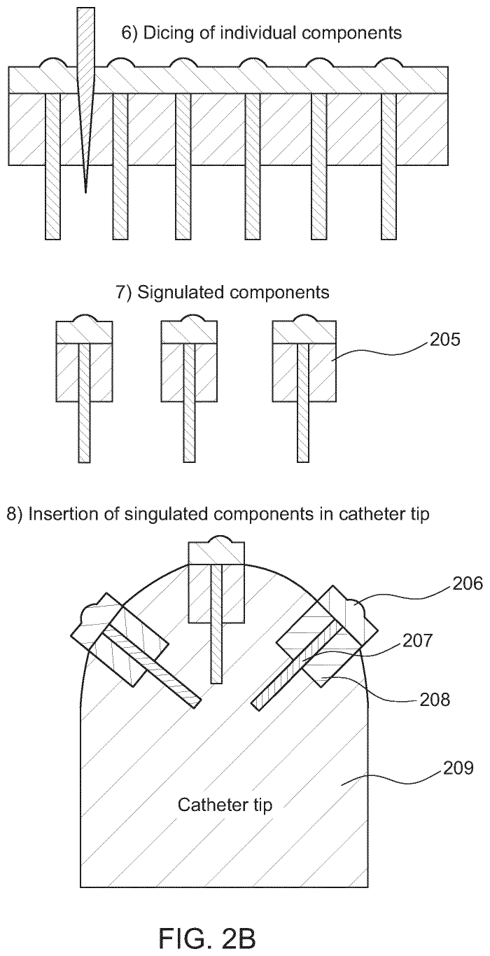

FIGS. 2A and 2B and exemplarily illustrates a method of manufacture of a catheter probe in accordance with some of the embodiments of the present invention. The method for manufacturing said optical fiber catheter probe comprises the following steps. A glass or silicon substrate 201 is selectively etched to form a plurality of vias 202 and an optical fiber 203 is inserted in each of said plurality of vias 202. The top surface comprising an excess of the optic fiber material is removed and the top surface of the glass substrate 201 is polished.

Further, a microlens array 204 having a plurality of microlenses, where each microlens is aligned with each corresponding optical fiber core 202 on said polished top surface of the glass substrate 101. Also, the aligned microlens array 204 is bonded on said polished top surface of the glass substrate. Thereafter, the microlens, optical fiber and glass substrate assembly is diced to obtain individual optical sensors 205. Finally, each of the individual optical sensor is inserted into a catheter tip of the optical fiber catheter probe.

Thereby, manufacturing a catheter probe having a catheter tip 209 and inserted within said catheter tip are a plurality of optical sensors 205. Each of said optical sensors comprises a microlens 206 attached to ferrule 208 and an optical fiber 207 running through a via of the ferrule 208 to optically couple said lens to a monitoring means or module (not shown).

In an embodiment, said optical fibers 207 has a diameter of about 50 microns and the core diameters of said optical fibers 207 are about 2-5 microns. In a preferred embodiment, the core diameters of said optical fibers 207 are around 2.5 microns. In a preferred embodiment, the vias have a diameter of 52-55 microns.

In an embodiment, said optical fibers are configured to operate at wavelengths of around 1310 nanometre.

The microlens array 204 is bonded to the top surface of the glass substrate 201 using an ultraviolet cure epoxy. Suitably the microlens array 204 is made up of silicon. In another preferred embodiment, the optical fiber 207 is a single mode fiber.

In another method for manufacturing a catheter probe comprises the following steps of: selectively etching a glass substrate to form a plurality of vias and inserting an optical fiber in each of said plurality of vias. Thereafter, forming a microlens on each optical fiber projecting through said plurality of vias, where each of said microlens on each optical fiber is formed using three dimensional polymer stereo-lithography or laser micromachining.

Further, glass substrate assembly comprising said glass substrate and said optical fiber and a microlens formed thereon, is diced to obtain individual optical sensors and each individual optical sensor is inserted into a catheter tip.

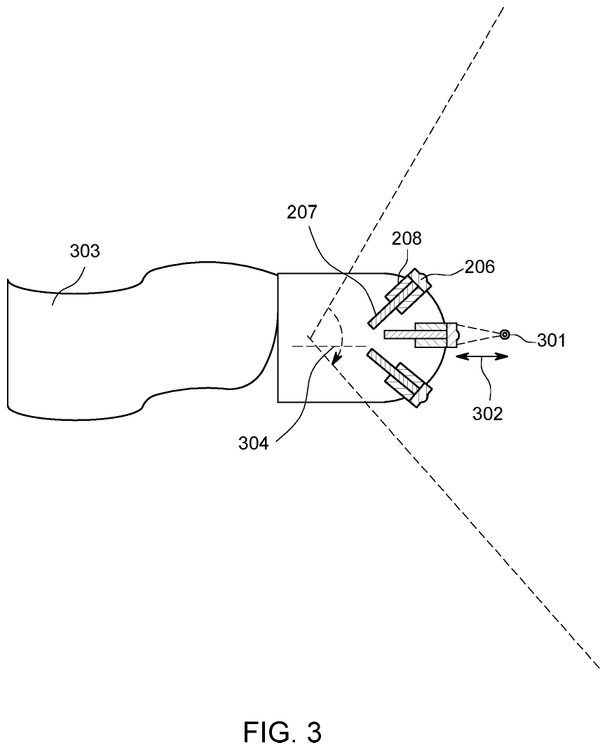

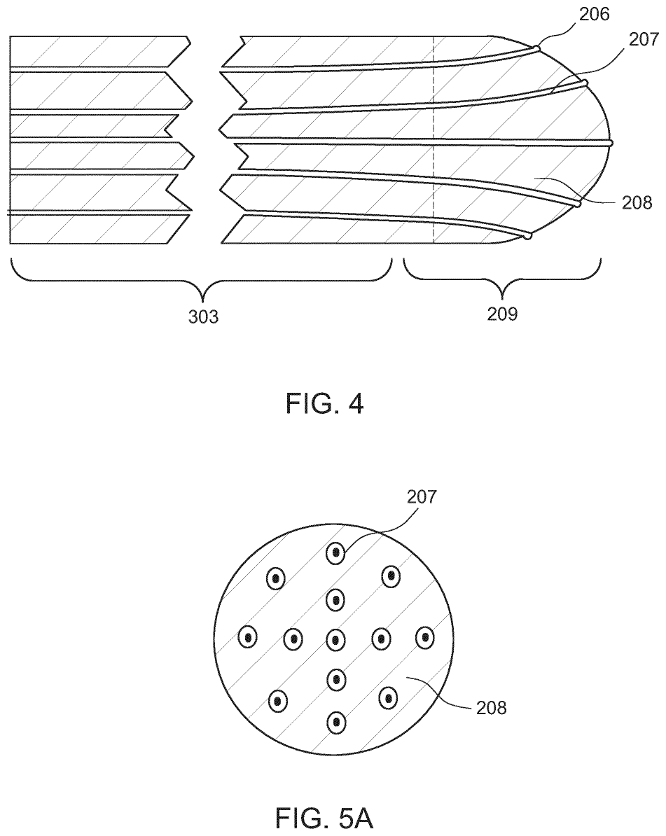

FIG. 3 exemplarily illustrates a sectional view of the catheter probe in accordance with some of the embodiments of the present invention and FIG. 4 exemplarily illustrates another sectional view of the catheter probe in accordance with some of the embodiments of the present invention. The catheter probe comprises a catheter tip 209 and a catheter body 303. The catheter tip comprises a plurality of channels or tunnels for housing a plurality of optical sensors 205. Each one of said plurality of optical sensors 205 comprises a ferrule 208 comprising a via, a microlens 206 aligned with said via and attached to said ferrule 208 and an optical fiber 207 for optically coupling said microlens 206 to a monitoring means.

The catheter body 303 houses said optical fibers 207 to enable optical coupling of said microlenses 206 to a monitoring means or module (not shown). The optical fibers 207 have a diameter of about 50 microns and the core diameters of said optical fibers 207 are about 2-5 microns. The optical fibers 207 can be configured to operate at wavelengths of around 1310 nanometre. In a preferred embodiment, the optical fiber 207 is a single mode fiber.

A narrow cladding single mode (SM) optical fiber 207 of the present invention is used as the mechanism to transport light form the source, along the catheter to the sensing tip. Said narrow clad fiber enables multiple fibers to be integrated in a single catheter instrument. Further, the narrow core region of the optical fibers 207 ensures minimum bending loss, which is especially critical when the catheter is inserted in the long and twisted vascular network of a subject.

The incorporation of micro lenses (optics) enables to focusing the sensing light beam to the (tissue) area of analysis (labelled as 301 in FIG. 3) as it exits the optical fiber. Also, the micro lens ensures an effective working distance (labelled as 302 in FIG. 3) from the fiber tip to the area of analysis. Typical working distances are in the order of 1-3 mm from the fiber tip.

In an embodiment, the microlenses comprises silicon microlenses for their high refractive index (compared to glass) which ensures the correct working distance can be achieved in water (as in most cases the lens may be inserted to a tissue region submerged in bodily fluids).

It will be appreciated that the plurality of optical sensors 205 provides a wide effective viewing angle (labelled as 304 in FIG. 3) and thereby maximizes the field of view for effective monitoring.

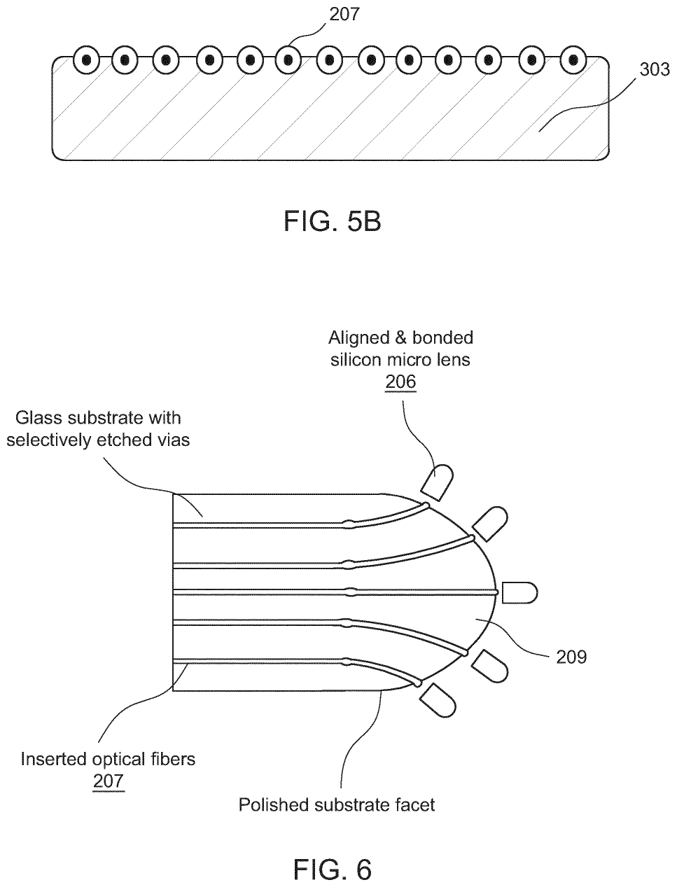

FIG. 5A exemplarily illustrates a sectional view of the catheter tip (cut along the broken line as shown in FIG. 4) of said catheter probe in accordance with some of the embodiments of the present invention and FIG. 5B exemplarily illustrates a sectional view of the catheter body of said catheter probe in accordance with some of the embodiments of the present invention.

The optical fibers 207 in the catheter tip 209 are radially arranged in accordance with some of the embodiments of the present invention. Further, in an embodiment 10-20 of individual sensor assemblies 205 may be used for a single catheter instrument.

Also, the proximal terminating end of the optical fiber body 303 as shown in FIG. 5B has optical fibers 207 arranged in a linear manner for easy optical coupling to a monitoring means.

FIG. 6 exemplarily illustrates a sectional view of the catheter body of said catheter probe in accordance with another embodiment of the invention. As shown, separate silicon micro lenses 206 can be aligned and attached to the component 209. The component comprises of the structured glass substrate with selectively etched vias. Optical fibers 207 are inserted into each of these vias. In FIG. 6, the distributed array of facets on the component, where the micro lenses 206 will be bonded, can be polished. The micro lenses are then aligned and attached to each facet using UV cure epoxy. In this embodiment, there is no need to dice the fully assembled component as it is fully integrated with fibers 207 and micro lenses 209 oriented in all sensing directions.

Narrow core optical fibers are also known as high optical index fibers are ideal for catheter-based sensing applications as they exhibit low optical bend loss. Catheters experience significant bending when inserted in the body (as they move along veins and arteries) and the high refractive index difference between the narrow waveguiding fiber core and outer cladding regions of the fiber ensure low optical loss due to fiber bending in the body.

A narrow core optical fiber have typical dimensions of 50-80 microns for the outer cladding region and inner core (waveguiding) dimensions of 3-5 microns. These dimensions ensure single mode waveguiding along the core at wavelengths in the range of 1300 nm to 1550 nm, while minimising bend loss. Different core sizes can be selected for alternative wavelengths, such as in the UV and visible wavelength regions. Lower operating wavelengths will require a smaller core diameter than that for the 1300 nm to 1550 nm wavelengths. The narrow cladding diameter of these fibers also enables multiple fibers to be arranged in a parallel configuration along the catheter body. This supports multi-point sensing.

Narrow core fibers can be difficult to align to micro optical elements such as the silicon micro lenses used to focus light from the output fiber to the region of sensing or interrogation. For example, standard optical fibers operating in the wavelength range of 1300 nm to 1550 nm have a core diameter on the range of 9-10 microns, compared to narrow core fibers with a diameter in the range 3-5 microns. This narrow core also limits the ability of the fiber core to receive or collect light returning from the sensing area. According to one aspect of the invention a mode adaptor design can be implemented to overcome this problem.

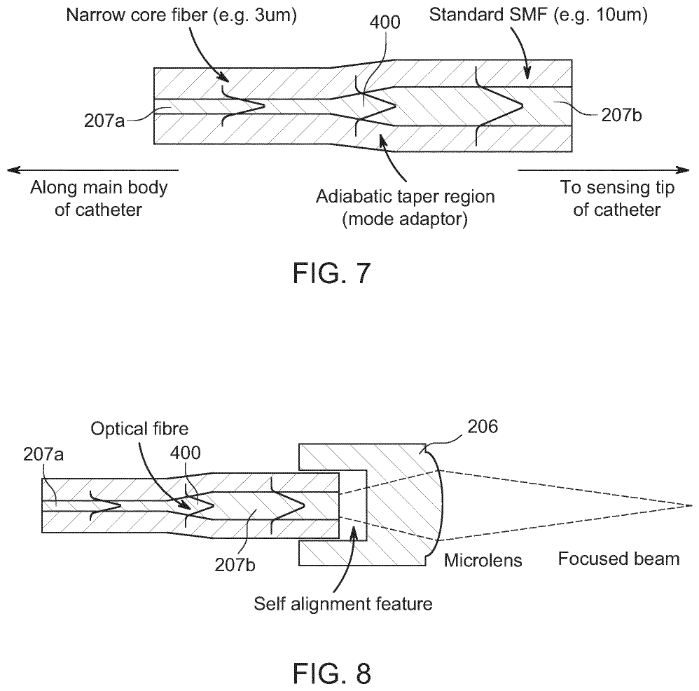

FIG. 7 illustrates a mode adaptor design in the optic fiber 207 to transition between a narrow core 207a and standard core 207b optical fiber. As illustrated in FIG. 7 a mode tapering element 400 which expands to the mode coming from the narrow core fiber 207a to a larger core fiber 207b. This can be achieved by fusing both fibers 207a, 207b together using a heating element, where a joint forms a tapered optical element which slowly transitions the narrow mode field size to the larger mode field size. This design feature is reversible, so the light signal returning from the sensing area can be more easily collected by the standard fiber and then tapered down to the narrow core diameter.

A relaxation of the microlens alignment tolerances due to the use of the fiber mode adaptor 400 also enables the use of microlens which incorporate self-aligning features. FIG. 8 illustrates how self-aligning features on the microlens 206 to facilitate passive alignment of the microlens 206 to a standard optical fiber 207b, avoiding the need for active alignment. The self-aligning features facilitate passive alignment of the microlens 206 to the standard optical fiber 207b, avoiding the need for active alignment where optical coupling power must be continuously monitored due the alignment process. This design enables a faster and more scalable manufacturing process.

In the specification the terms “comprise, comprises, comprised and comprising” or any variation thereof and the terms “include, includes, included and including” or any variation thereof are considered to be totally interchangeable and they should all be afforded the widest possible interpretation and vice versa.

A person skilled in the art would appreciate that the above invention provides a robust and economical solution to the problems identified in the prior art.

The invention is not limited to the embodiments hereinbefore described but may be varied in both construction and detail.

1. A catheter probe, comprising:
a catheter tip, said catheter tip comprising a plurality of channels which house a plurality of optical sensors, each plurality of optical sensors comprising:
a ferrule comprising a central micro via;
an optical fiber passed through said micro via;
a microlens attached to said ferrule and aligned to said optical fiber; and
a catheter body configured to house said optical fibers and connected to a monitoring means.

2. The catheter of claim 1, wherein said optical fibers has a diameter of about 50 microns and the core diameter of said optical fibers is about 2-5 microns.

3. The catheter of claim 1, where said vias have a diameter of about 52-55 microns.

4. The catheter of claim 1, wherein said optical fibers are configured to operate at wavelengths of around 1310 nanometre.

5. The catheter of claim 1, wherein the optical fiber is a single mode fiber.

6. The catheter of claim 1, wherein the optical fiber has a narrow cladding and high numerical aperture.

7. The catheter of claim 1, wherein said optical fibre comprises a dimensioned tapered channel to define a fiber mode adapter.

8. The catheter of claim 7 wherein the fiber comprises a narrow core fiber to a larger core fiber wherein the tapered channel expands a mode from the narrow core fiber to the larger core fiber.

9. A method for manufacturing a catheter probe, comprising:
etching a glass substrate to form a plurality of vias;
inserting an optical fiber in each of said plurality of vias;
polishing a top surface of the glass substrate;
aligning a micro-lens array, where each microlens of the microlens array is aligned with each via on said polished top surface of the glass substrate;
bonding said aligned microlens array on said polished top surface of the glass substrate;
dicing said microlens, optical fiber and glass substrate to obtain individual optical sensors; and
inserting each optical sensor into a catheter tip.

10. The method of claim 9, wherein said optical fibers has a diameter of about 50 microns and the core diameter of said optical fibers is about 2-5 microns.

11. The method of claim 9, where said vias have a diameter of about 52-55 microns.

12. The method of claim 9, wherein said optical fibers are configured to operate at wavelengths of around 1310 nanometre.

13. The method of claim 9, wherein said microlens array is bonded to the top surface of the glass substrate using an ultraviolet cure epoxy.

14. The method of claim 9, wherein the microlens array comprises silicon.

15. The method of claim 9, wherein the optical fiber is a single mode fiber.

16. A method for manufacturing a catheter probe, comprising:
etching a glass substrate to form a plurality of vias;
inserting an optical fiber in each of said plurality of vias;
forming a microlens on each optical fiber projecting through said plurality of vias;
dicing a glass substrate assembly comprising said glass substrate and said optical fiber and a microlens formed thereon, to obtain individual optical sensors; and
inserting each optical sensor into a catheter tip.

17. The method of claim 16, wherein said optical fibers has a diameter of about 50 microns and the core diameter of said optical fibers is about 2-5 microns.

18. The method of claim 16, wherein said optical fibers are configured to operate at wavelengths of around 1310 nanometre.

19. The method of claim 16, wherein said forming of a microlens on each optical fiber comprises three dimensional polymer stereo-lithography or laser micromachining.

20. The method of any of claim 16, wherein the optical fiber is a single mode fiber.

21. A method for manufacturing a catheter probe, comprising:
etching a glass substrate to form a plurality of vias;
inserting an optical fiber in each of said plurality of vias;
polishing a top surface of the glass substrate;
aligning a micro-lens array, where each microlens of the microlens array is aligned with each via on said polished top surface of the glass substrate;
bonding said aligned microlens array on said polished top surface of the glass substrate; and
inserting each optical sensor into a catheter tip.
